(19) United States
(12) Patent Application Publication
(10) Pub. No.: US 2018/0271827 A1
(43) Pub. Date: Sep. 27, 2018

(54) CANNABINOID COMPOSITIONS AND METHODS OF MAKING

(71) Applicant: Pasenture, Inc., Palo Alto, CA (US)

(72) Inventors: JACOB FOSTER HEIMARK, Palo Alto, CA (US); Justin Thomas Gwin, Palo Alto, CA (US); William Royden McFarland, Palo Alto, CA (US)

(21) Appl. No.: 15/762,391

(22) PCT Filed: Sep. 23, 2016
(86) PCT No.: PCT/US16/53346

§ 371 Date: Mar. 22, 2018

Related U.S. Application Data
(60) Provisional application No. 62/232,187, filed on Sep. 24, 2015.

(51) Int. Cl. A61K 31/352; B01D 11/02; A23G 4/06; A23L 33/105; A61K 31/05; A61K 47/56

(52) U.S. Cl. CPC A61K 31/352; B01D 11/0288; A23G 4/068; A23V 2002/00; A61K 31/05; A61K 47/56; A23L 33/105

(57) ABSTRACT

A food additive comprising cannabinoids but lacking at least in part the taste and aroma associated with cannabis while retaining the psychoactive and medicinal properties thereof is provided for as well as methods of making.

### BACKGROUND

Cannabis containing foods or edibles are becoming increasingly popular with the legalization of cannabis in certain states. Some reports estimate that edibles comprise more than 40% of the recreational cannabis market in Colorado.

Concentrates derived from cannabis plants can be referred to as cannabis oil, budder, wax, kief, or shatter. They can be significantly more potent than regular hashish or cannabis flowers. Cannabis concentrate extraction depends on the solubility of the cannabinoid and other active ingredients of the cannabis plant.

Most edibles are made with lipid, glycerin or ethanol extractions. These extractions, however, have odor and taste elements from the cannabis that are retained and readily detectable in edible compositions. To some consumers, these odors and/or tastes are undesirable. Terpenes are compounds that are attributable to at least some of the objectionable odor and taste of cannabis extracts. Previous attempts at removing terpenes and other volatile compounds from cannabinoids in cannabis, while retaining the psychoactive properties of the cannabinoids, have been unsuccessful.

Thus there remains a need to develop psychoactive and medicinal compositions for use in edibles that appeal to a wider range of consumers and methods of making them.

### SUMMARY

Certain aspects disclosed herein are drawn to methods of producing a cannabinoid-bonded polymer composition. In certain embodiments, such methods comprise removing at least about 90% of the solvent from a cannabinoid solvent absolute obtained from a dewaxed cannabinoid concentrate concrete. In certain embodiments, such methods also comprise stripping at least of portion of the terpenes from a cannabinoid solvent absolute obtained from a dewaxed cannabinoid concentrate concrete. And, in certain embodiments, such methods also comprise decarboxylating at least a portion of the cannabinoids contained in a cannabinoid solvent absolute obtained from a dewaxed cannabinoid concentrate concrete. These processes produce a stripped and decarboxylated cannabinoid absolute. The stripped and decarboxylated cannabinoid absolute is then dissolved in a polymer-engrossing solution, thereby producing a cannabinoid-bonded polymer composition. In certain embodiments, the polymer-engrossing solution comprises food grade polymer and a solvent. In certain embodiments, the solvent of the polymer engrossing solution comprises ethanol. In certain embodiments, the food grade polymer of the polymer engrossing solution comprises shellac. In certain embodiments, the cannabinoid-bonded polymer composition comprises pharmaceutical lactose and/or xylitol.

In certain embodiments, the cannabinoid solvent absolute obtained from a dewaxed cannabinoid concentrate concrete is obtained by: i) dissolving a cannabinoid concentrate concrete in a solvent at a temperature of between about 13° C. to about 82° C. to produce a cannabinoid concrete solution; ii) cooling the cannabinoid concrete solution to a temperature of between about 10° C. and about −125° C.; and iii) separating at least a portion of the impurities from the cooled cannabinoid concrete solution to produce a cannabinoid solvent absolute. In certain embodiments, the impurities are separated by filtration. In certain embodiments the filtration is through a filter of about 75 microns or finer. In certain embodiments, the filtration is through a filter of between about 15 microns and about 75 microns. In certain embodiment, the impurities are separated by precipitation. In certain embodiments, the precipitation is performed at temperature of between about −100° C. and about 15° C.

In certain embodiments disclosed herein, the solvent of the cannabinoid solvent absolute has a boiling point of from about 20° C. to about 100° C. In certain embodiments, the solvent of the cannabinoid solvent absolute is an alcohol or nontoxic hydrocarbon solvent. In certain embodiments, the solvent of the cannabinoid solvent absolute is selected from the group consisting of methyl ether, butane, hexane, propane, ethanol, and carbon dioxide. In certain embodiments, the solvent of the cannabinoid solvent absolute is selected from the group consisting of anhydrous ethanol, methyl ether, and butane.

In certain embodiments, methods comprise the separate steps of first removing the solvent from the cannabinoid solvent absolute to form a cannabinoid absolute and then stripping the terpenes from the cannabinoid absolute and decarboxylating the cannabinoids in the cannabinoid absolute to produce a stripped and decarboxylated cannabinoid absolute.

In certain embodiments, removing the solvent comprises heating the cannabinoid solvent absolute to a temperature of at least about 30° C. to about the boiling point of the cannabinoid in the cannabinoid solvent absolute with the lowest boiling point. In certain embodiments, removing the solvent comprises heating the cannabinoid solvent absolute to a temperature of between about 30° C. to about 90° C.

In certain embodiments, stripping the terpenes and decarboxylating the cannabinoids is performed at a temperature of between about 65° C. and about the boiling point of the cannabinoid in the cannabinoid solvent absolute with the lowest boiling point. In certain embodiments, stripping the terpenes and decarboxylating the cannabinoids is performed at a temperature of between about 65° C. and about 125° C. In certain embodiments, stripping the terpenes and decarboxylating the cannabinoids is done for a duration of time of between about 10 minutes to about 128 hours or of between about 7 hours to about 24 hours.

In certain embodiments, at least about 50% of the terpenes are stripped from those present in the cannabinoid solvent absolute. In certain embodiments, at least about 80% of the cannabinoids present in the stripped and decarboxylated cannabinoid absolute are decarboxylated.

In certain embodiments, the stripped and decarboxylated cannabinoid absolute is dissolved in the polymer engrossing solution at a temperature of between about 30° C. and 110° C.

In certain embodiments one or more terpenes is added to the cannabinoid-bonded polymer composition. In certain embodiments, the one or more terpenes is selected from the group consisting of menthol, menthone, menthoxypropanediol, menthylacetate, myrcenol, citronellal, senchone, and thioterpineol

In certain embodiments, the cannabinoid-bonded polymer composition exhibits psychoactive cannabinoid activity.

In certain embodiments of the methods, the cannabis odor of the cannabinoid-bonded polymer composition is less than that of the starting cannabinoid concentrate concrete containing the same concentration of cannabinoids, as determined by a blind odor test group containing a statistically significant number of subjects. In certain embodiments, the cannabis odor of the cannabinoid-bonded polymer composition, containing a cannabinoid concentration of at least about 1% by weight, is imperceptible on average to subjects in an odor test group, containing a statistically significant number of subjects, who are unaware that the cannabinoid-bonded polymer composition contains cannabinoids. In certain embodiments, the cannabis odor of the cannabinoid-bonded polymer composition, containing a cannabinoid concentration of at least about 1% by weight, is imperceptible to at least about 60% of subjects in an odor test group, containing a statistically significant number of subjects, who are unaware that the cannabinoid-bonded polymer composition contains cannabinoids.

In certain embodiments of the methods, the cannabis taste of the cannabinoid-bonded polymer composition is less than in the starting cannabinoid concentrate concrete containing the same concentration of cannabinoids, as determined by a blind taste test group containing a statistically significant number of subjects. In certain embodiments, the cannabis taste of the cannabinoid-bonded polymer composition, containing a cannabinoid concentration of at least about 1% by weight, is imperceptible on average to subjects in a taste test group, containing a statistically significant number of subjects, who are unaware whether the cannabinoid-bonded polymer composition contains cannabinoids. In certain embodiments, the cannabis taste of the cannabinoid-bonded polymer composition, containing a cannabinoid concentration of at least about 1% by weight, is imperceptible to at least about 60% of subjects in a taste test group, containing a statistically significant number of subjects, who are unaware whether the cannabinoid-bonded polymer composition contains cannabinoids.

In certain embodiments, the cannabis odor of an edible product that exhibits psychoactive cannabinoid activity, comprising the cannabinoid-bonded polymer composition, is imperceptible on average or imperceptible to at least about 60% to subjects in an odor test group, containing a statistically significant number of subjects, who are unaware whether the edible product contains cannabinoids. In certain embodiments, the cannabis taste of an edible product that exhibits psychoactive cannabinoid activity, comprising the cannabinoid-bonded polymer composition, is imperceptible on average or imperceptible to at least about 60% of subjects in a taste test group, containing a statistically significant number of subjects, who are unaware whether the edible product contains cannabinoids.

In certain embodiments of the methods, the cannabinoid-bonded polymer composition comprises from about 0.1% to about 22% by weight of cannabinoids. In certain embodiments, the cannabinoid-bonded polymer composition comprises from about 2% to about 18% by weight of cannabinoids.

Certain aspects are drawn to a cannabinoid-bonded polymer composition made by any of the methods disclosed herein. In certain embodiments, the cannabinoid-bonded polymer composition comprises about 0.1% to about 22% by weight of cannabinoids. In certain embodiments, the cannabinoid-bonded polymer composition comprises about 2% to about 18% by weight of cannabinoid. In certain embodiments, the cannabinoid-bonded polymer composition comprises ethanol. In certain embodiments, the cannabinoid-bonded polymer composition comprises shellac. In certain embodiments, the cannabinoid-bonded polymer composition comprises pharmaceutical lactose and/or xylitol.

Certain aspects are drawn to a cannabinoid-bonded polymer composition comprising about 0.1% to about 22% by weight of a cannabinoid, a food grade polymer, and a solvent. In certain embodiments, the cannabinoid-bonded polymer composition comprises about 2% to about 18% by weight of a cannabinoid. In certain embodiments, the solvent is ethanol. In certain embodiments, the food grade polymer is shellac. In certain embodiments, the cannabinoid-bonded polymer composition comprises pharmaceutical lactose and/or xylitol.

In certain embodiments, a cannabinoid-bonded polymer composition contains a cannabinoid concentration of at least about 1% by weight and the cannabis odor of the composition is imperceptible on average to or is imperceptible to at least about 60% of subjects in an odor test group, containing a statistically significant number of subjects, who are unaware that the cannabinoid-bonded polymer composition contains cannabinoids. In certain embodiments, a cannabinoid-bonded polymer composition contains a cannabinoid concentration of at least about 1% by weight and the cannabis taste of the composition is imperceptible on average to or is imperceptible to at least about 60% of subjects in a taste test group, containing a statistically significant number of subjects, who are unaware whether the cannabinoid-bonded polymer composition contains cannabinoids.

In certain embodiments, the cannabis odor of an edible product that exhibits psychoactive cannabinoid activity, comprising a cannabinoid-bonded polymer composition, is imperceptible on average to or is imperceptible to at least about 60% of subjects in an odor test group, containing a statistically significant number of subjects, who are unaware that the edible product contains cannabinoids. In certain embodiments, the cannabis taste of an edible product that exhibits psychoactive cannabinoid activity, comprising a cannabinoid-bonded polymer composition, is imperceptible on average to or is imperceptible to at least about 60% of subjects in a taste test group, containing a statistically significant number of subjects, who are unaware whether the edible product contains cannabinoids.

Certain aspects are drawn to methods for delivering a cannabinoid to a subject, the method comprising placing an edible product that comprises a cannabinoid-bonded polymer composition described herein into the mouth of the subject. In certain embodiments, a cannabinoid in the cannabinoid-bonded polymer composition is absorbed sublingually, mucosally, or sublingually and mucosally. In certain embodiments, the edible product is a chewing gum. In certain embodiments, the method produces psychoactive symptoms associated with cannabinoids in the subject.

Certain aspects are drawn to an edible product comprising a cannabinoid-bonded polymer composition described herein. In certain embodiments, the edible product has psychoactive activity associated with cannabinoids. In certain embodiments, the cannabis odor from a cannabinoid-bonded polymer composition in the edible product is imperceptible on average to or imperceptible to at least about 60% of subjects in an odor test group, containing a statistically significant number of subjects, who are unaware whether the edible product contains cannabinoids. In certain embodiments, the cannabis taste from a cannabinoid-bonded polymer composition in the edible product is imperceptible on average to or imperceptible to at least about 60% of subjects in a taste test group, containing a statistically significant number of subjects, who are unaware whether the edible composition contains cannabinoids. In certain embodiments, the edible product is a chewing gum.

### BRIEF DESCRIPTION OF THE DRAWINGS/FIGURES

FIG. 1. FIG. 1 depicts decarboxylation of 9-tetrahydrocannabinol (THC) and cannabidiol (CBD).

### DETAILED DESCRIPTION

### Definitions

To the extent necessary to provide descriptive support, the subject matter and/or text of the appended claims is incorporated herein by reference in their entirety. It will be understood by all readers of this written description that the exemplary embodiments described and claimed herein may be suitably practiced in the absence of any recited feature, element or step that is, or is not, specifically disclosed herein.

Throughout this disclosure, the term “a” or “an” entity refers to one or more of that entity; for example, “a terpene,” is understood to represent one or more “terpenes”. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related. Numeric ranges are inclusive of the numbers defining the range. Even when not explicitly identified by “and any range in between,” where a list of values is recited, e.g., “1, 2, 3, or 4,” the disclosure specifically includes any range in between the values, e.g., “1 to 3,” “1 to 4,” “2 to 4,” etc. The headings provided herein are not limitations of the various aspects or aspects of the disclosure, which can be had by reference to the specification as a whole. Accordingly, the terms defined immediately below are more fully defined by reference to the specification in its entirety.

As used herein, “cannabis” refers to a well-known genus of flowering plants that includes at least three different species: Cannabis sativa; Cannabis indica; and Cannabis ruderalis. When a composition is referred to as “containing cannabis” or the like, it is understood that the composition has one or more ingredients derived from a cannabis plant but may not actually contain a whole plant.

As used herein, “cannabinoid-bonded polymer” means a large molecule of repeated subunits, i.e., the “polymer,” that is bonded with a molecule that acts on cannabinoid receptors of cells. In certain embodiments, a “cannabinoid-polymer polymer composition” comprises a cannabinoid(s) bonded to a polymer side chain(s) where both the polymer and the cannabinoids are in a nonpolar solvent.

As used herein, “cannabinoids,” “cannabinoid compounds,” and the like, refer to molecules that act on the “endocannabinoid system.” The endocannabinoid system comprises a class of cell membrane receptors that are part of the G protein-coupled receptor superfamily.

As used herein, “to strip,” “stripping,” “stripped,” and the like refers to the process of separating one compound from one or more other compounds.

As used herein, “cannabinoid concentrate concrete” refers to a composition that contains cannabinoid molecules in a concentration of no less than about 30% cannabinoid by weight.

As used herein, “dewaxing” or “to dewax” refers to the process of removing some or all of the fats, lipids, waxes, and/or soaps from an unrefined compound containing cannabinoids. “Dewaxed” refers to a composition that has undergone the dewaxing process. The term “winterized” as used herein is synonymous with the term “dewaxed.”

As used herein, “cannabis odor” (or “smell”) refers to the fragrance associated with one or more volatilized chemical compounds produced by plants of the genus Cannabis that humans or other animals perceive by the sense of olfaction.

As used herein, “cannabis taste” (or “flavor”) refers to the taste of substances produced by plants of the genus Cannabis that are perceived by humans or other animals by the sense of gustation. One of ordinary skill in the art will recognize the relationship between the perception of taste and smell.

As used herein, “shellac” refers to a resin secreted by the female lac bug on trees such as in the forests of India and Thailand. It can be processed and sold as dry flakes that can be dissolved in a solvent such as ethanol to make liquid shellac. Liquid shellac has uses including as a colorant, food glaze, and wood finish.

### Cannabinoid-Bonded Polymer Composition

The present disclosure provides for cannabinoid-containing compositions. These compositions include cannabinoid-bonded polymer compositions. In certain embodiments, the cannabinoid-bonded polymer compositions can be included in edible products. In certain embodiments, a cannabinoid-bonded polymer composition is one producible by any method for making such product disclosed herein. By “producible” it is meant that the composition can be obtained using a method described herein but could also be obtained by a different method, so long as a method described herein will also produce it. In certain embodiments, a cannabinoid-bonded polymer composition is characterized by actually being produced by a method described herein, whether or not it could be produced by another method. It is understood that even in the absence of a specific chemical composition, which may be ascertained by methods of chemical analysis that are routine in the art, the embodiments cover any compositions that are producible by methods disclosed herein.

Cannabinoids are believed to be the predominant compounds in cannabis responsible for the psychoactive properties associated with cannabis use. The amount of cannabinoids in a cannabinoid-bonded polymer composition can vary depending on a number of factors such as the amount of cannabinoids present in the original cannabis plant and the methods by which the cannabinoids are isolated. In certain embodiments, the concentration of cannabinoids in a cannabinoid-bonded polymer composition is between about 0.1% to about 25% by weight. In certain embodiments, the concentration of cannabinoids in a cannabinoid-bonded polymer composition is about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or any range in-between. In certain embodiments, the concentration of cannabinoids in a cannabinoid-bonded polymer composition is from about 1%, 2%, or 3% to about 25% or from about 1%, 2%, or 3% to about 18%. In certain embodiments, the concentration of cannabinoids in a cannabinoid-bonded polymer composition is at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, 30%, 35%, 40%, or 50%. In certain embodiments, the concentration of cannabinoids in a cannabinoid-bonded polymer composition is not more than about 0.2%, 0.3%, 0.4%, 0.5%, 0.6%, 0.7%, 0.8%, 0.9%, 1.0%, 2.0%, 3.0%, 4.0%, 5.0%, 6.0%,7.0%, 8.0%, 9.0%, 10%, 11%, 12%, 13%, 14%, 15%, 16%, 17%, 18%, 19%, 20%, 21%, 22%, 23%, 24%, 25%, 30%, 35%, 40%, or 50%.

In certain embodiments, the amount of decarboxylation of cannabinoids in a cannabinoid-bonded polymer composition is about 25%, 30%, 40%, 50%, 60%, 70%, 75%, 80%, 90%, 95%, 98%, 99%, 100%, or any range in-between. In certain embodiments, at least about 25%, 30%, 40%, 50%, 60%, 70%, 75%, 80%, 90%, 95%, 98%, 99%, or 100% of the cannabinoids are decarboxylated. In certain embodiments, not more than about 25%, 30%, 40%, 50%, 60%, 70%, 75%, 80%, 90%, 95%, 98%, or 99% of the cannabinoids are decarboxylated. In certain embodiments, the amount is at least about 80%.

In addition to cannabinoids, a cannabinoid-bonded polymer composition comprises a solvent and a food grade polymer. In certain embodiments, the solvent is one or more of ethanol, acetone, ethyl ether, and/or methyl ether. In certain embodiments, the solvent is ethanol. In certain embodiments, the amount of solvent in the cannabinoid-bonded polymer composition is about 1%, 5%, 10%, 15%, 20%, 25%, 30%, 35%, 40%, 45%, 50%, or any range in-between, by weight. In certain embodiments, the amount of solvent in the cannabinoid-bonded polymer composition is not more than about 1%, 5%, 10%, 15%, 20%, 25%, 30%, 35%, 40%, 45%, or 50%. In certain embodiments, flavors can be added to the cannabinoid-bonded polymer composition and such flavors are considered part of the solvent, and thus would increase the concentration of solvent. In certain embodiments, the remaining percentage or substantially all of the remaining percentage of the cannabinoid-bonded polymer composition other than the solvent is food grade polymer. In certain embodiments, the amount of food grade polymer is about 99%, 95%, 90%, 85%, 80%, 75%, 70%, 65%, 60%, 55%, 50%, 45%, 40%, 35%, 30%, 25% or any range in-between, by weight. In certain embodiments, the amount of food grade polymer is at least about 25%, 30%, 35%, 40%, 45%, 50%, 55%, 60%, 65%, 70%, 75%, 80%, 85%, 90%, or 95%. In certain embodiments the food grade polymer is shellac. In certain embodiments, a cannabinoid-bonded polymer composition comprises other components suitable for use in an edible food product such as sweeteners, flavorings, preservatives, texture modifiers, fiber, etc. In certain embodiments, a cannabinoid-bonded polymer composition includes pharmaceutical lactose and/or xylitol.

Shellac can be dewaxed for use via solvent extraction. In some embodiments, it is dissolved in a solvent such as ethanol. Impurities and wax can be removed by, for example filtration, and the shellac solution can be decolorized by the addition of activated carbon. Following removal of the activated carbon, such as by additional filtration, the solvent can be removed by evaporation, for example on a thin film evaporator and the shellac recovered. Removal of the solvent increases the concentration of the shellac solution. A hot molten shellac mass can be obtain that can be cast into a film. Once cool, the film can be broken into shellac flakes.

In certain embodiments, a cannabinoid-bonded polymer composition has a low terpene to cannabinoid ratio. In certain embodiments the terpene to cannabinoid ratio is lower than the ratio of the source material from which the cannabinoid-bonded polymer composition is obtained. In certain embodiments, the cannabinoid-bonded polymer composition has a terpene to cannabinoid ration of: about 3 mg terpene: 100 mg cannabinoid; 2 mg terpene: 100 mg cannabinoid; or about 1.5 mg terpene: 100 mg cannabinoid; or about 1 mg terpene: 100 mg cannabinoid; or about 0.5 mg terpene: 100 mg cannabinoid; or about 0.1 mg terpene: 100 mg cannabinoid; or about 0.05 mg terpene: 100 mg cannabinoid; or about 0.01 mg terpene: 100 mg cannabinoid; or any range in between. In certain embodiments, the cannabinoid-bonded polymer composition has a terpene to cannabinoid ration that is lower than: about 3 mg terpene: 100 mg cannabinoid; or about 2 mg terpene: 100 mg cannabinoid; or about 1.5 mg terpene: 100 mg cannabinoid; or about 1 mg terpene: 100 mg cannabinoid; or about 0.5 mg terpene: 100 mg cannabinoid; or about 0.1 mg terpene: 100 mg cannabinoid; or about 0.05 mg terpene: 100 mg cannabinoid. In certain embodiments, the cannabinoid-bonded polymer composition has a terpene to cannabinoid ration that is not greater than: about 3 mg terpene: 100 mg cannabinoid; or about 2 mg terpene: 100 mg cannabinoid; or about 1.5 mg terpene: 100 mg cannabinoid; or about 1 mg terpene: 100 mg cannabinoid; or about 0.5 mg terpene: 100 mg cannabinoid; or about 0.1 mg terpene: 100 mg cannabinoid; or about 0.05 mg terpene: 100 mg cannabinoid; or about 0.01 mg terpene: 100 mg cannabinoid.

### Odor

In certain embodiments, a cannabinoid-bonded polymer composition has a cannabis odor that is decreased, diminished, lessened, and the like compared to a cannabis source or cannabis extract from which it was derived, for a given amount of cannabinoids. In certain embodiments, a cannabinoid-bonded polymer composition has a cannabis odor that is decreased, diminished, lessened, and the like compared to a cannabis source or cannabis extract from which it was derived, for a given amount of cannabinoids, as determined by a decrease in odor-causing volatile compounds in the cannabinoid-bonded polymer composition.

In certain embodiments, the amount of odor-causing volatile compounds in a composition, such as before and after making a cannabinoid-bonded polymer composition, can be determined by measuring the amount (e.g., by using a gas chromatograph (GC) or high performance liquid chromatography (HPLC)) of terpenes and/or cannabinoids. The terpene to cannabinoid ratio can be measured independently of other molecules. In certain embodiments, the terpene to cannabinoid ratio can be decreased (e.g., the amount of terpenoids decreased disproportionally in comparison to the change in the amount of cannabinoids) by the process of forming a cannabinoid-bonded polymer composition.

In certain embodiments, compounds including terpenes, e.g.: myrcene; pinene; humulene; limonene; and terpineol, that account in part for a composition having a cannabis odor, have been stripped and less than about 1%, 2%, 3%, 4%, 5%, 6%, 7%, 8%, 9%, 10%, 11%, 12%, 13%, 14%, 14%, or 15% of such terpenes remain in the cannabinoid-bonded polymer composition in comparison to the starting cannabinoid concentrate concrete. In some embodiments, less than about 1%, 2%, 3%, 4%, 5%, 6%, 7%, 8%, 9%, 10%, 11%, 12%, 13%, 14%, 14%, or 15% of such terpenes remain in the cannabinoid-bonded polymer composition in comparison to the starting cannabinoid concentrate concrete, for a given amount of cannabinoids.

In certain embodiments, a cannabinoid-bonded polymer composition has a cannabis odor that is faint, not easily detected, not easily perceived, nearly undetectable, nearly imperceptible, undetectable, and/or imperceptible to a person not trained to detect cannabis. In certain embodiments, a cannabinoid-bonded polymer composition has a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis odor that is faint, not easily detected, not easily perceived, nearly undetectable, nearly imperceptible, undetectable, and/or imperceptible to a person not trained to detect cannabis.

In certain embodiments, a cannabinoid-bonded polymer composition has a cannabis odor that is imperceptible on average to subjects in an odor test group, containing a statistically significant number of subjects, who are unaware whether the cannabinoid-bonded polymer composition contains cannabinoids. In certain embodiments, a cannabinoid-bonded polymer composition has a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis odor that is imperceptible on average to subjects in an odor test group, containing a statistically significant number of subjects, who are unaware whether the cannabinoid-bonded polymer composition contains cannabinoids.

In certain embodiments, a cannabinoid-bonded polymer composition has a cannabis odor that is imperceptible to at least about 10%, 20%, 25%, 30%, 40%, 50%, 60%, 70%, 75%, 80%, 90%, 95%, 96%, 97%, 98%, 99%, or 100% subjects in an odor test group, containing a statistically significant number of subjects, who are unaware whether the cannabinoid-bonded polymer composition contains cannabinoids. In certain embodiments, a cannabinoid-bonded polymer composition has a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis odor that is imperceptible to at least about 10%, 20%, 25%, 30%, 40%, 50%, 60%, 70%, 75%, 80%, 90%, 95%, 96%, 97%, 98%, 99%, or 100% of subjects in an odor test group, containing a statistically significant number of subjects, who are unaware whether the cannabinoid-bonded polymer composition contains cannabinoids.

Cannabis odor can be detected and characterized as strong, easily detected, weak, mild, faint, not easily detected, not easily perceived, nearly undetectable, nearly imperceptible, undetectable, and/or imperceptible by a person with a normal sense of smell. Cannabis odor can be compared between multiple compositions (e.g., one smells more strongly of cannabis than another) by a person with a normal sense of smell. Unless otherwise specified herein, a person with a normal sense of smell is not someone trained to detect cannabis or otherwise considered to have a heightened sense of smell. To provide statistical rigor, any of the above attributes of cannabis odor can be determined by an odor test group of multiple subjects. Although the number of subjects in an odor test group may vary, there should be enough subjects so that the results of any determination or comparison are considered statistically significant by one of ordinary skill in the art. In certain tests, the subjects of the test group are unaware that the composition they are smelling contains cannabis. In certain tests, the subjects of the test group are unaware that any composition they are asked to smell could contain cannabis.

### Taste

In certain embodiments, a cannabinoid-bonded polymer composition has a cannabis taste that is decreased, diminished, lessened, and the like compared to a cannabis source or cannabis extract from which it was derived, for a given amount of cannabinoids. In certain embodiments, a cannabinoid-bonded polymer composition has a cannabis taste that is decreased, diminished, lessened, and the like compared to a cannabis source or cannabis extract from which it was derived, as determined by a decrease in flavor-causing volatile compounds in the cannabinoid-bonded polymer composition, for a given amount of cannabinoids.

The amount of flavor-causing volatile compounds in a composition can be measured in a number of ways. For example, volatile compounds can be measured by gas chromatography (GC) or high performance liquid chromatography (HPLC).

In certain embodiments, compounds including terpenes, e.g.: myrcene; pinene; humulene; limonene; and terpineol, that account in part for a composition having a cannabis flavor, have been stripped and less than about 1%, 2%, 3%, 4%, 5%, 6%, 7%, 8%, 9%, 10%, 11%, 12%, 13%, 14%, 14%, or 15% of such terpenes remain in the cannabinoid-bonded polymer composition in comparison to the starting cannabinoid concentrate concrete. In some embodiments, less than about 1%, 2%, 3%, 4%, 5%, 6%, 7%, 8%, 9%, 10%, 11%, 12%, 13%, 14%, 14%, or 15% of such terpenes remain in the cannabinoid-bonded polymer composition in comparison to the starting cannabinoid concentrate concrete, for a given amount of cannabinoids.

In certain embodiments, a cannabinoid-bonded polymer composition has a cannabis taste that is faint, not easily detected, not easily perceived, nearly undetectable, nearly imperceptible, undetectable, and/or imperceptible to a person not trained to detect cannabis. In certain embodiments, a cannabinoid-bonded polymer composition contains a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis taste that is faint, not easily detected, not easily perceived, nearly undetectable, nearly imperceptible, undetectable, and/or imperceptible to a person not trained to detect cannabis.

In certain embodiments, a cannabinoid-bonded polymer composition has a cannabis taste that is imperceptible on average to subjects in a taste test group, containing a statistically significant number of subjects, who are unaware whether the cannabinoid-bonded polymer composition contains cannabinoids. In certain embodiments, a cannabinoid-bonded polymer composition contains a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis taste that is imperceptible on average to subjects in a taste test group, containing a statistically significant number of subjects, who are unaware whether the cannabinoid-bonded polymer composition contains cannabinoids.

In certain embodiments, a cannabinoid-bonded polymer composition has a cannabis taste that is imperceptible to at least about 10%, 20%, 25%, 30%, 40%, 50%, 60%, 70%, 75%, 80%, 90%, 95%, 96%, 97%, 98%, 99%, or 100% subjects in a taste test group, containing a statistically significant number of subjects, who are unaware whether the cannabinoid-bonded polymer composition contains cannabinoids. In certain embodiments, a cannabinoid-bonded polymer composition contains a cannabinoid concentration of at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30% by weight, and has a cannabis taste that is imperceptible to at least about 10%, 20%, 25%, 30%, 40%, 50%, 60%, 70%, ‘75%, 80%, 90%, 95%, 96%, 97%, 98%, 99%, or 100% of subjects in a taste test group, containing a statistically significant number of subjects, who are unaware whether the cannabinoid-bonded polymer composition contains cannabinoids.

Cannabis taste can be detected and characterized as strong, easily detected, weak, mild, faint, not easily detected, not easily perceived, nearly undetectable, nearly imperceptible, undetectable, and/or imperceptible by a person with a normal sense of taste. Cannabis taste can be compared between multiple compositions (e.g., one tastes more strongly of cannabis than another) by a person with a normal sense of taste. Unless otherwise specified herein, a person with a normal sense of taste is not someone trained to detect cannabis or otherwise considered to have a heightened sense of taste. To provide statistical rigor, any of the above attributes of cannabis odor can be determined by a taste test group of multiple subjects. Although the number of subjects in a taste test group may vary, there should be enough subjects so that the results of any determination or comparison are considered statistically significant by one of ordinary skill in the art. In certain tests, the subjects of the test group are unaware whether any particular composition they are tasting contains cannabis, although for ethical reasons, they would be aware that the compositions they are tasting could contain cannabis.

### Psychoactive Effects

Use of cannabis is associated with psychoactive effects (referred to herein interchangeably as psychoactive “symptoms” of cannabis or of cannabinoids and also referred to when describing a product as having cannabinoid psychoactive “activity,” and the like) which are widely attributed to the cannabinoid compounds produced by cannabis plants.

These effects are colloquially referred to as “getting high” or “getting stoned,” but also include symptoms of medical significance such as increased appetite and decreased nausea.

In certain embodiments, psychoactive cannabinoid activity is associated with a cannabinoid-bonded polymer composition having at least about 80% decarboxylation of the cannabinoids.

### Edibles

Certain embodiments provide for edible products comprising a cannabinoid-bonded polymer composition as described anywhere herein. In certain embodiments, an edible product comprises about 0.1% to about 100% by weight of a cannabinoid-bonded polymer composition as described anywhere herein. In certain embodiments, an edible product comprises about 0.1%, 0.25%, 0.5%, 0.75%, 1%, 2%, 3%, 4%, 5%, 6%, 7%, 8%, 9%, 10%, 15%, 20%, 25%, 30%, 40%, 50%, 60%, 70%, 80%, 90%, 100%, or any range in between of a cannabinoid-bonded polymer composition as described anywhere herein. An edible product can be any product that is suitable, e.g., non-toxic, for placing into the mouth of a human, whether ingested, absorbed, or only chewed or sucked on and at least a portion discarded, etc. Illustrative examples of edible products include chewing or bubble gums, mints, suckers, jawbreakers, lozenges, hard candies, gummy candies, taffies, chocolates, brownies, cookies, crackers, granola or meal replacement bars, powdered drink mixes, soft drinks, flavored water and water flavorings or additives, sports drinks, tinctures, smokeless inhalation powders, honey, syrup, spreads, and dissolving strips.

In certain embodiments, the concentration of cannabinoids in an edible product is between about 0.1% to about 25% by weight. In certain embodiments the concentration of cannabinoids in an edible product is about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or any range in between. In certain embodiments, the concentration of cannabinoids in an edible product is at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, or 25%. In certain embodiments, the concentration of cannabinoids in an edible product is not more than about 0.2%, 0.3%, 0.4%, 0.5%, 0.6%, 0.7%, 0.8%, 0.9%, 1.0%, 2.0%, 3.0%, 4.0%, 5.0%, 6.0%,7.0%, 8.0%, 9.0%, 10%, 11%, 12%, 13%, 14%, 15%, 16%, 17%, 18%, 19%, 20%, 21%, 22%, 23%, 24%, or 25%.

In certain embodiments, the amount of decarboxylation of cannabinoids in an edible composition comprising a cannabinoid-bonded polymer composition as described elsewhere herein is about 25%, 30%, 40%, 50%, 60%, 70%, 75%, 80%, 90%, 95%, 98%, 99%, 100%, or any range in-between. In certain embodiments, at least about 25%, 30%, 40%, 50%, 60%, 70%, 75%, 80%, 90%, 95%, 98%, 99%, or 100% of the cannabinoids are decarboxylated. In certain embodiments, not more than about 25%, 30%, 40%, 50%, 60%, 70%, 75%, 80%, 90%, 95%, 98%, 99%, or 100% of the cannabinoids are decarboxylated. In certain embodiments, the amount is at least about 80%.

In certain embodiments, the amount of cannabinoids and/or amount of decarboxylation of the cannabinoids in an edible product is enough to produce noticeable psychoactive effects associated with cannabis in a subject consuming at least a recommended amount of the edible product. Generally, a recommended amount is an amount that will produce psychoactive effects but not so great as to cause undesirable side effects or toxic effects.

In certain embodiments, an edible product that produces psychoactive cannabinoid effects comprising a cannabinoid-bonded polymer composition has a cannabis odor that is faint, not easily detected, not easily perceived, nearly undetectable, nearly imperceptible, undetectable, and/or imperceptible to a person not trained to detect cannabis as described in detail elsewhere herein. In certain embodiments, an edible product that produces psychoactive cannabinoid effects comprising a cannabinoid-bonded polymer composition has a cannabis odor that is imperceptible on average to subjects in an odor test group, containing a statistically significant number of subjects, who are unaware whether the cannabinoid-bonded polymer composition contains cannabinoids. In certain embodiments, an edible product that produces psychoactive cannabinoid effects comprising a cannabinoid-bonded polymer composition has a cannabis odor that is imperceptible to at least about 10%, 20%, 25%, 30%, 40%, 50%, 60%, 70%, 75%, 80%, 90%, 95%, 96%, 97%, 98%, 99%, or 100% subjects in an odor test group, containing a statistically significant number of subjects, who are unaware whether the cannabinoid-bonded polymer composition contains cannabinoids.

In certain embodiments, an edible product that produces psychoactive cannabinoid effects comprising a cannabinoid-bonded polymer composition has a cannabis taste that is faint, not easily detected, not easily perceived, nearly undetectable, nearly imperceptible, undetectable, and/or imperceptible to a person not trained to detect cannabis as described in detail elsewhere herein. In certain embodiments, an edible product that produces psychoactive cannabinoid effects comprising a cannabinoid-bonded polymer composition has a cannabis taste that is imperceptible on average to subjects in a taste test group, containing a statistically significant number of subjects, who are unaware whether the cannabinoid-bonded polymer composition contains cannabinoids. In certain embodiments, an edible product that produces psychoactive cannabinoid effects comprising a cannabinoid-bonded polymer composition has a cannabis taste that is imperceptible to at least about 10%, 20%, 25%, 30%, 40%, 50%, 60%, 70%, 75%, 80%, 90%, 95%, 96%, 97%, 98%, 99%, or 100% subjects in a taste test group, containing a statistically significant number of subjects, who are unaware whether cannabinoid-bonded polymer composition contains cannabinoids.

### Method of Producing a Cannabinoid-Bonded Polymer Composition

Certain aspects disclosed herein are drawn to methods of producing a cannabinoid-bonded polymer composition. In certain embodiments, a starting material for the method is a cannabinoid solvent absolute which has been obtained by dewaxing a cannabinoid concentrate such as obtained from a cannabis extract from a cannabis plant, such as a cannabinoid concentrate concrete. Illustrative examples of cannabinoid concentrates include hydrocarbon extracted hash oil concentrates, carbon dioxide extracted concentrates, pure trichome dry sift concentrates, cold water extracted concentrates, or other equivalent cannabinoid extracts from cannabis plants. Illustrative examples of useful extractions include hexane based extraction, ethanol based extraction, carbon dioxide based extraction, heat pressed extraction (a.k.a. rosin), butane based extraction (e.g., produces butane hash oil (BHO)), and purified cannabinoid crystals or purified cannabinoid salts.

For hydrocarbon extracted hash oil concentrates, suitable hydrocarbons include butane, hexane, propane, diesel, kerosene, gasoline, naphtha and ethanol. These concentrates can be produced by applying liquid hydrocarbon to plant material. During this process, the hydrocarbon extracts the cannabinoids from the plant material.

For carbon dioxide extracted concentrates, subcritical carbon dioxide extraction is produced by forcing liquid carbon dioxide through plant material at various pressures to force cannabinoids to separate from plant cells.

Pure trichome dry sift concentrates can be produced by mechanically forcing trichomes, the main cannabinoid producing parts of plants in the cannabis genus, through a series of different grades of micron filters to separate plant materials from cannabinoids.

Cold water extracted concentrates can be produced where under cold water, trichomes and cannabinoids increase in density, making them easier to separate using a method similar to the pure trichome dry sift concentrate.

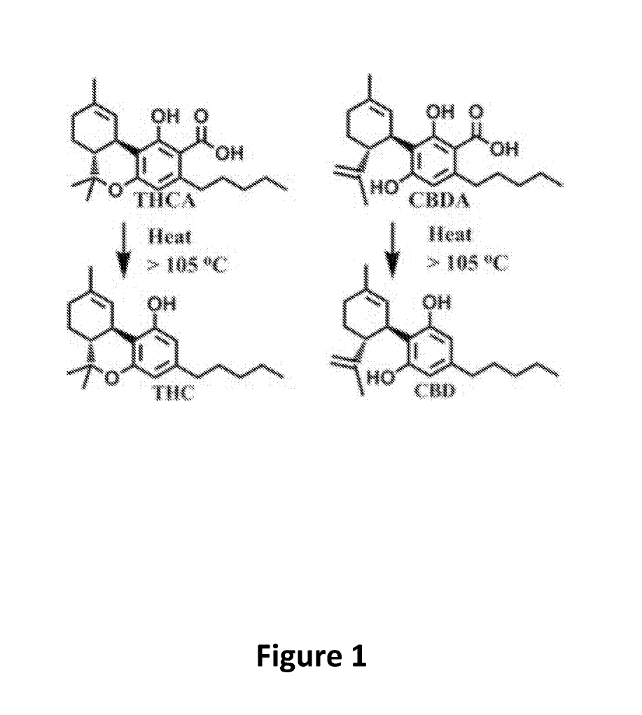

In certain embodiments, at least about 50%, 60%, 70%, 80%, 90%, 95%, 98%, 99%, or 100% of the solvent of a cannabinoid solvent absolute is removed from the cannabinoid solvent absolute to produce a cannabinoid absolute. In certain embodiments, at least a portion of the terpenes from the cannabinoid solvent absolute are stripped away (for the purposes of this disclosure, to avoid confusion with the term “remove” in reference to the solvent, the term “strip” or “stripping” is used to refer to the separation of terpenes from a composition). And, in certain embodiments, at least a portion of the cannabinoids from the cannabinoid solvent absolute are decarboxylated. FIG. 1 shows decarboxylation of the cannabinoids 9-tetrahydrocannabinol (THC) and cannabidiol (CBD) wherein carboxyl groups (-COOH) are removed from THC and CBD. Removal of the solvent, stripping of terpenes, and decarboxylation of cannabinoids can occur simultaneously, separately, or partially simultaneously and partially separately. For example, the solvent may be removed at a first temperature for a certain period of time and after a certain amount of the solvent has been removed, the temperature can be changed (such as to a higher temperature) for a period of time to strip the terpenes and/or decarboxylate the cannabinoids.

The temperature at which the solvent of the cannabinoid solvent absolute is removed can vary, such as depending on the type of solvent used. In certain embodiments, the solvent has a boiling point of from about 20° C. to about 100° C. In certain embodiments, the solvent is removed at a temperature that is least about the boiling point of the solvent. It is understood that the “boiling point” referred to anywhere herein is the boiling point at the atmospheric pressure under which the step is performed. Thus, while a solvent may have, for example, a “sea level” boiling point, the “boiling point” temperature chosen to remove the solvent may be significantly different if the solvent removal step is performed under a vacuum. Generally, a liquid in a vacuum environment has a lower boiling point than when the liquid is at atmospheric pressure. In certain embodiments, the solvent is removed at a temperature of about 5° C., 10° C., 15° C., 20° C., 25° C., 30° C., 40° C., 50° C., 60° C., 70° C., 80° C., 90° C., 100° C., or any range in between. In certain embodiments, the solvent is removed at a temperature of at least about 5° C., 10° C., 15° C., 20° C., 25° C., 30° C., 40° C., 50° C., 60° C., 70° C., 80° C., 90° C., or 100° C. In certain embodiments, the solvent is removed at a temperature of not greater than about 10° C., 15° C., 20° C., 25° C., 30° C., 40° C., 50° C., 60° C., 70° C., 80° C., 90° C., or 100° C. In certain embodiments, the solvent is removed at a temperature that is at least about 5° C., 10° C., 15° C., 20° C., 25° C., 30° C., 40° C., 50° C., or 100° C. higher than the boiling point of the solvent. In certain embodiments, the solvent has a boiling point of from about 20° C. to about 100° C. In certain embodiments, the solvent has a boiling point of about 20° C., 25° C., 30° C., 35° C., 40° C., 45° C., 50° C., 55° C., 60° C., 65° C., 70° C., 75° C., 80° C., 85° C., 90° C., 95° C., 100° C., or any range in between. In certain embodiments, the solvent has a boiling point of greater than about 20° C., 25° C., 30° C., 35° C., 40° C., 45° C., 50° C., 55° C., 60° C., 65° C., 70° C., 75° C., 80° C., 85° C., 90° C., 95° C., or 100° C. In certain embodiments, the solvent has a boiling point of less than about 25° C., 30° C., 35° C., 40° C., 45° C., 50° C., 55° C., 60° C., 65° C., 70° C., 75° C., 80° C., 85° C., 90° C., 95° C., or 100° C.

In certain embodiments, the solvent is removed by heating the cannabinoid solvent absolute to a temperature of at least about 20° C., 25° C., 30° C., 35° C., 40° C., 45° C., 50° C., 55° C., 60° C., 65° C., 70° C., 75° C., 80° C., 85° C., 90° C., 95° C., or 100° C. to about the boiling point temperature of the cannabinoid in the cannabinoid solvent absolute with the lowest boiling point.

It will be understood that the removal of solvent is dependent on both temperature and time. Thus, at any of the temperatures disclosed herein, the step of solvent removal is for a duration of time sufficient to remove at least about 50%, 60%, 70%, 80%, 90%, 95%, 98%, 99%, or 100% of the solvent of a cannabinoid solvent absolute. It will also be understood that the removal of solvent can be influence by the atmospheric pressure under which the removal occurs. For example, in certain embodiments, the removal of solvent is performed under a vacuum or partial vacuum, which in some cases can reduce the temperature and/or time required to remove a certain amount of solvent.

In certain embodiments, the solvent of the cannabinoid solvent absolute is an alcohol, a nontoxic hydrocarbon solvent, or a mixture thereof. In certain embodiments, the solvent of the cannabinoid solvent absolute is one or more of methyl ether, butane, hexane, propane, ethanol, and carbon dioxide. In certain embodiments, the solvent of the cannabinoid solvent absolute is one or more of anhydrous ethanol, methyl ether, and butane.

In certain embodiments, the cannabinoid solvent absolute and/or cannabinoid absolute is heated to strip terpenes and decarboxylate the cannabinoids. It should be understood that the terms “heated” or “heating” or “cooled” or “cooling” and the like are in reference generally to whether thermal energy is added or removed to maintain a certain temperature, and not necessarily in relation to the temperature of the preceding step. For example, a solution heated in a first step can be additionally referred to as heated in an immediately following step, even though the temperature in the second step is lower than the temperature in the first step. At temperatures around ambient temperature, such as room temperature (e.g., about 20° C. to 25° C.), a step may be referred to either as heating or cooling. Reference to either heating or cooling in a method step should not be interpreted as a limitation as to the temperature of preceding or following steps, unless otherwise specified.

The temperature at which terpenes are stripped can vary, such as depending on the boiling point of the terpenes. The temperature at which terpenes are stripped can also take into account the boiling point of the cannabinoids in a composition comprising both terpenes and cannabinoids. In general, the temperature at which the terpenes are stripped is a temperature that is at least about the boiling point of the terpene present with the lowest boiling point. One of ordinary skill in the art will recognize that as the temperature is raised above the boiling point of other terpenes present, a higher percentage of the terpenes can be stripped. In certain embodiments, the temperature at which the terpenes are stripped is at least about the boiling point of the terpene present with the highest boiling point. One of ordinary skill in the art will also recognize the parameters of time and atmospheric pressure can influence the temperature chose to perform the stripping of terpenes. Table A lists representative terpenes and their boiling points and properties or predicted properties.

TABLE A

| | Approximate | | |
|---|---|---|---|
| Terpenoid Essential Oil | Boiling Point | | Properties |
| ß-myrcene | 166-168 | degrees C. | Analgesic. Antiinflammatory, Antibiotic, |
| | | | Antimutagenic |
| ß-caryophyllene | 119 | degrees C. | Antiinflammatory, Cytoprotective (gastric mucosa), |
| | | | Antimalarial |
| d-limonene | 177 | degrees C. | Potential cannabinoid agonist, Immune potentiator, |
| | | | Antidepressant, Antimutagenic |
| linalool | 198 | degrees C. | Sedative, Antidepressant, Anxiolytic, Immune |
| | | | potentiator |
| pulegone | 224 | degrees C. | Potential memory booster, AChE inhibitor, Sedative, |
| | | | Antipyretic |
| 1,8-cineole (eucalyptol) | 176 | degrees C. | AChE inhibitor, Increases cerebral, blood flow, |
| | | | Stimulant, Antibiotic, Antiviral, Antiinflammatory, |
| | | | Antinociceptive |
| a-pinene | 156 | degrees C. | Antiinflammatory, Bronchodilator, Stimulant, |
| | | | Antibiotic, Antineoplastic, AChE inhibitor |
| a-terpineol | 217-218 | degrees C. | Sedative, Antibiotic, AChE inhibitor, Antioxidant, |
| | | | Antimalarial |
| terpineol-4-ol | 209 | degrees C. | AChE inhibitor, Antibiotic |
| p-cymene | 117 | degrees C. | Antibiotic, Anticandidal, AChE inhibitor |

In certain embodiments, the temperature at which the terpenes are stripped is a temperature that does not destroy or drive off an undesirable amount of cannabinoids. By an undesirable amount, it is meant an amount that would significantly decrease or eliminate the psychoactive activity of the resultant cannabinoid-bonded polymer composition. In certain embodiments, the temperature at which the terpenes are stripped is not more than about the boiling point of the cannabinoid present with the lowest boiling point. In certain embodiments, the temperature at which the terpenes are stripped is less than about the boiling point of the cannabinoid present with the lowest boiling point. In certain embodiments, the temperature at which the terpenes are stripped is not more than about the boiling point of the cannabinoid present, which is meant to be retained, with the lowest boiling point. In certain embodiments, the temperature at which the terpenes are stripped is less than about the boiling point of the cannabinoid present, which is meant to be retained, with the lowest boiling point. Table B lists representative cannabinoids and their boiling points and properties or predicted properties.

TABLE B

| | Approximate | |
|---|---|---|
| Cannabinoid | Boiling Point | Properties |
| 9-tetrahydrocannabinol (THC) | 157 degrees C. | Euphoriant, Analgesic, |
| | | Antiinflammatory, Antioxidant, |
| | | Antiemetic |
| cannabidiol (CBD) | 160-180 degrees C. | Anxiolytic, Analgesic, Antipsychotic, |
| | | Antiinflammatory, Antioxidant, |
| | | Antispasmodic |
| Cannabinol (CBN) | 185 degrees C. | Oxidation, breakdown, product, |
| | | Sedative, Antibiotic |
| cannabichromene (CBC) | 220 degrees C. | Antiinflammatory, Antibiotic, |
| | | Antifungal |
| cannabigerol (CBG) | | Antiinflammatory, Antibiotic, |
| | | Antifungal |
| ?-8-tetrahydrocannabinol (?-8- | 175-178 degrees C. | Resembles ?-9-THC, Less psychoactive, |
| THC) | | More stable Antiemetic |
| tetrahydrocannabivarin (THCV) | 220 degrees C. | Analgesic, Euphoriant |

Table C lists other representative flavonoid and phytosterol components derived from cannabis plants and their boiling points and properties or predicted properties.

TABLE C

| Flavonoid or | Approximate | |
|---|---|---|
| Phytosterol | Boiling Point | Properties |
| apigenin | 178 degrees C. | Anxiolytic, Antiinflammatory, Estrogenic |
| quercetin | 250 degrees C. | Antioxidant, Antimutagenic, Antiviral, |
| | | Antineoplastic |
| cannflavin A | 182 degrees C. | COX inhibitor, LO inhibitor |
| ß-sitosterol | 134 degrees C. | Antiinflammatory, 5-a-reductase, inhibitor |

In certain embodiments, the temperature at which the terpenes are stripped is between about 25° C., 30° C., 40° C., 50° C., 60° C., 65° C., 70° C., 80° C., 90° C., 100° C., and about the boiling point of the cannabinoid in the cannabinoid solvent absolute with the lowest boiling point. In certain embodiments, the temperature at which the terpenes are stripped is about 25° C., 30° C., 40° C., 50° C., 60° C., 65° C., 70° C., 80° C., 90° C., 100° C., 125° C., 150° C., 175° C., 200° C., and any range in between. In certain embodiments, the temperature at which the terpenes are stripped is at least about 25° C., 30° C., 40° C., 50° C., 60° C., 65° C., 70° C., 80° C., 90° C., 100° C., 125° C., 150° C., 175° C., or 200° C. In certain embodiments, the temperature at which the terpenes are stripped is not more than about 30° C., 40° C., 50° C., 60° C., 65° C., 70° C., 80° C., 90° C., 100° C., 125° C., 125° C., 150° C., 175° C., or 200° C.

In certain embodiments and at any of the stripping temperatures disclosed herein, the terpenes can be stripped for a duration of time of about 1 minute, 2 minutes, 3 minutes, 5 minutes, 10 minutes, 20 minutes, 30 minutes, 45 minutes, 60 minutes, 120 minutes, 3 hours, 6 hours, 7 hours, 12 hours, 18 hours, 24 hours, 36 hours, 48 hours, 72 hours, 96 hours, 120 hours, 128 hours, or any range in-between. In certain embodiments and at any of the stripping temperatures disclosed herein, the terpenes can be stripped for a duration of time of at least about 1 minute, 2 minutes, 3 minutes, 5 minutes, 10 minutes, 20 minutes, 30 minutes, 45 minutes, 60 minutes, 120 minutes, 3 hours, 6 hours, 7 hours, 12 hours, 18 hours, 24 hours, 36 hours, 48 hours, 72 hours, 96 hours, 120 hours, or 128 hours. In certain embodiments and at any of the stripping temperatures disclosed herein, the terpenes can be stripped for a duration of time of not more than about 1 minute, 2 minutes, 3 minutes, 5 minutes, 10 minutes, 20 minutes, 30 minutes, 45 minutes, 60 minutes, 120 minutes, 3 hours, 6 hours, 7 hours, 12 hours, 18 hours, 24 hours, 36 hours, 48 hours, 72 hours, 96 hours, 120 hours, or 128 hours. In certain embodiments and at any of the stripping temperatures disclosed herein, the terpenes can be stripped for a duration of time of from about 7 hours to 24 hours.

In certain embodiments, about 25%, 30%, 40%, 50%, 60%, 70%, 75%, 80%, 90%, 95%, 98%, 99%, 100%, or any range in-between of the terpenes are stripped from those present in the cannabinoid solvent absolute. In certain embodiments, at least about 25%, 30%, 40%, 50%, 60%, 70%, 75%, 80%, 90%, 95%, 98%, 99%, or 100% of the terpenes are stripped from those present in the cannabinoid solvent absolute. In certain embodiments, not more than about 25%, 30%, 40%, 50%, 60%, 70%, 75%, 80%, 90%, 95%, 98%, 99%, or 100% of the terpenes are stripped from those present in the cannabinoid solvent absolute.

The psychoactive properties of cannabinoids are believed to be at least in part attributed to decarboxylation of cannabinoid compounds. Decarboxylation can occur under the same or similar conditions (e.g., temperature, time, atmospheric pressure) used to strip terpenes described in detail elsewhere herein. Higher temperatures result in faster decarboxylation but higher temperatures increase the likelihood of vaporizing cannabinoids and/or altering undesirably altering their chemical structure. In certain embodiments, the time, temperature, and/or atmospheric pressure is identical to that used to strip terpenes as described herein. In certain embodiments, the time, temperature, and/or atmospheric pressure can be altered to drive maximum decarboxylation of the cannabinoids, even at the expense of stripping a higher amount of terpenes, or in excess of the temperature, time, and/or atmospheric pressure needed to remove a certain amount of terpenes.

In certain embodiments, about 25%, 30%, 40%, 50%, 60%, 70%, 75%, 80%, 90%, 95%, 98%, 99%, 100%, or any range in-between of the cannabinoids in the stripped and decarboxylated cannabinoid absolute are decarboxylated. In certain embodiments, at least about 25%, 30%, 40%, 50%, 60%, 70%, 75%, 80%, 90%, 95%, 98%, 99%, or 100% of the cannabinoids in the stripped and decarboxylated cannabinoid absolute are decarboxylated. In certain embodiments, not more than about 25%, 30%, 40%, 50%, 60%, 70%, 75%, 80%, 90%, 95%, 98%, 99%, or 100% of the cannabinoids in the stripped and decarboxylated cannabinoid absolute are decarboxylated.

In certain embodiments, it is contemplated a method or composition wherein the amount of terpenes and decarboxylated cannabinoids in the stripped and decarboxylated cannabinoid absolute is a combination of any of the amounts of terpenes and decarboxylated cannabinoids disclosed herein.

The solvent can be removed before the steps of stripping terpenes and decarboxylating the cannabinoids, thus forming a cannabinoid absolute from which the terpenes are stripped and the cannabinoids are decarboxylated to form a stripped and decarboxylated cannabinoid absolute. The solvent can also be removed simultaneously with the stripping of terpenes and decarboxylation of cannabinoids or the processes can overlap such that cannabinoid solvent absolute is converted to a stripped and decarboxylated cannabinoid absolute without the producer performing separate steps of solvent removal and then stripping and decarboxylation.

Regardless of the method of converting the cannabinoid solvent absolute into a stripped and decarboxylated cannabinoid absolute, in certain embodiments, the stripped and decarboxylated cannabinoid absolute is dissolved in a polymer-engrossing solution that comprises at least a food-grade polymer and a solvent. In certain embodiments, the solvent is one or more of ethanol, acetone, ethyl ether, and/or methyl ether. In certain embodiments, the solvent is ethanol. In certain embodiments the food grade polymer is shellac.

Dissolving the stripped and decarboxylated cannabinoid absolute in the polymer-engrossing solution produces a cannabinoid-bonded polymer composition. In certain embodiments, other components suitable for use in an edible food product are added such as sweeteners, flavorings, preservatives, texture modifiers, fiber, etc. In certain embodiments, the method includes adding to a cannabinoid-bonded polymer composition pharmaceutical lactose and/or xylitol.

In certain embodiments, the resulting concentration of cannabinoids in a cannabinoid-bonded polymer composition is between about 0.1% to about 25% by weight. In certain embodiments, the resulting concentration of cannabinoids in a cannabinoid-bonded polymer composition is about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, 30% or any range in-between. In certain embodiments, the resulting concentration of cannabinoids in a cannabinoid-bonded polymer composition is at least about 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, 20%, 21%, 22%, 23%, 24%, 25%, or 30%. In certain embodiments, the resulting concentration of cannabinoids in a cannabinoid-bonded polymer composition is not more than about 0.2%, 0.3%, 0.4%, 0.5%, 0.6%, 0.7%, 0.8%, 0.9%, 1.0%, 2.0%, 3.0%, 4.0%, 5.0%, 6.0%,7.0%, 8.0%, 9.0%, 10%, 11%, 12%, 13%, 14%, 15%, 16%, 17%, 18%, 19%, 20%, 21%, 22%, 23%, 24%, 25%, or 30%.

In certain embodiments, the amount of decarboxylation of cannabinoids in a cannabinoid-bonded polymer composition is about 25%, 30%, 40%, 50%, 60%, 70%, 75%, 80%, 90%, 95%, 98%, 99%, 100%, or any range in-between. In certain embodiments, at least about 25%, 30%, 40%, 50%, 60%, 70%, 75%, 80%, 90%, 95%, 98%, 99%, or 100% of the cannabinoids are decarboxylated. In certain embodiments, not more than about 25%, 30%, 40%, 50%, 60%, 70%, 75%, 80%, 90%, 95%, 98%, 99%, or 100% of the cannabinoids are decarboxylated. In certain embodiments, the amount is at least about 80%.

The temperature at which the stripped and decarboxylated cannabinoid absolute is dissolved in the polymer-engrossing solution can depend, such as on the surface area available on the food grade polymer used. For example, generally smaller shellac fakes or a liquid shellac solution will not require as high a temperature to dissolve as larger shellac flakes. In certain embodiments, the stripped and decarboxylated cannabinoid absolute is dissolved in the polymer-engrossing solution at a temperature of about 5° C., 10° C., 20° C., 25° C., 30° C., 40° C., 50° C., 60° C., 70° C., 80° C., 90° C., 100° C., 110° C., 120° C., 125° C., or any range in-between. In certain embodiments, the stripped and decarboxylated cannabinoid absolute is dissolved in the polymer-engrossing solution at a temperature of at least about 5° C., 10° C., 20° C., 25° C., 30° C., 40° C., 50° C., 60° C., 70° C., 80° C., 90° C., 100° C., 110° C., 120° C., or 125° C. In certain embodiments, the stripped and decarboxylated cannabinoid absolute is dissolved in the polymer-engrossing solution at a temperature of not more than about 10° C., 20° C., 25° C., 30° C., 40° C., 50° C., 60° C., 70° C., 80° C., 90° C., 100° C., 110° C., 120° C., or 125° C.

Although in certain embodiments, certain terpenes are stripped from the composition in order, for example, to decrease or remove odor and taste associated with cannabis, certain terpenes or a certain amount of terpenes may be desirable in a cannabinoid-bonded polymer composition or edible product made therewith to act as carriers for cannabinoids and increase the psychoactivity and bio-availability of the cannabinoids. In certain embodiments, one or more terpenes can be added to the cannabinoid-bonded polymer composition. Illustrative examples of terpenes that can be added include menthol, menthone, menthoxypropanediol, menthylacetate, myrcenol, citronella, senchone, and/or thioterpineol. These added terpenes may come from any source. In certain embodiments, the terpenes are obtained by distilling the terpenes that are stripped from the original cannabinoid solvent absolute. Thus, the amount and/or composition of terpenes in the cannabinoid-bonded polymer composition can be adjusted to optimize flavor, aroma, and/or cannabinoid delivery.

Although generally the cannabinoid-bonded polymer composition would be added to other ingredients to form an edible product and would not be consumed on its own, it is still disclosed herein that the cannabinoid-bonded polymer composition produces psychoactive cannabinoid symptoms.

In certain embodiments of the methods for producing a cannabinoid-bonded polymer composition disclosed herein, the cannabis odor and/or cannabis taste of the resultant cannabinoid-bonded polymer composition is any as described in detail elsewhere herein.

Dewaxing

Methods of dewaxing a cannabinoid extracts such as cannabis concentrates including cannabis concentrate concretes to form a cannabinoid solvent absolute are known in the art. In certain embodiments, a cannabinoid solvent absolute is obtained from a cannabinoid concentrate concrete by: i) dissolving a cannabinoid concentrate concrete in a solvent to produce a cannabinoid concrete solution; ii) cooling the cannabinoid concrete solution; and iii) separating at least a portion of the impurities from the cooled cannabinoid concrete solution to produce a cannabinoid solvent absolute. The properties and types of solvents that can be used to dissolve a cannabinoid concentrate concrete and thus form a component of the cannabinoid solvent absolute are described elsewhere herein.

In certain embodiments, the cannabinoid concentrate concrete is dissolved at a temperature of about 10° C., 13° C., 20° C., 25° C., 30° C., 40° C., 50° C., 60° C., 70° C., 80° C., 82° C., 90° C., 100° C., or any range in between, to produce a cannabinoid concrete solution. In certain embodiments, the cannabinoid concentrate concrete is dissolved at a temperature of at least about 10° C., 13° C., 20° C., 25° C., 30° C., 40° C., 50° C., 60° C., 70° C., 80° C., 82° C., 90° C., 100° C., to produce a cannabinoid concrete solution. In certain embodiments, the cannabinoid concentrate concrete is dissolved at a temperature of not greater than about 20° C., 25° C., 30° C., 40° C., 50° C., 60° C., 70° C., 80° C., 82° C., 90° C., 100° C., to produce a cannabinoid concrete solution.

In certain embodiments, the cannabinoid concrete solution is cooled to a temperature of about −150° C., −125° C., −100° C., −75° C., −50° C., −25° C., −10° C., 0° C., 10° C., 20° C., 25° C., or any range in between. In certain embodiments, the cannabinoid concrete solution is cooled to a temperature of less than about −150° C., −125° C., −100° C., −75° C., −50° C., −25° C., −10° C., 0° C., 10° C., 20° C., or 25° C. In certain embodiments, the cannabinoid concrete solution is cooled to a temperature not lower than about −150° C., −125° C., −100° C., −75° C., −50° C., −25° C., −10° C., 0° C., 10° C., or 20° C.

Once cooled, at least a portion of the impurities in the cannabinoid concrete solution can be separated out to produce a cannabinoid solvent absolute. Illustrative examples of impurities that can be removed include lipids, plant waxes, chlorophyll, polymers, plant materials (e.g., methyl cellulose), pesticides, herbicides, fertilizers, nutrients, water, and microorganisms. Methods of separation are well known in the art. In certain embodiments, the impurities are separated by filtration. It will be understood that the finer the filter, the smaller the impurity size that can be removed. However, finer filters are more prone to clogging and may limit the rate at which a solution can be passed through the filter. Also, the temperature of the cannabinoid concrete solution can in part determine the viscosity of the solution and therefore the rate at which the solution can be passed through a filter. Thus, depending on the temperature, a finer or coarser filter may be more or less useful. Generally, at higher temperatures a finer filter may be used and at cooler temperatures a larger filter size may operate better. In certain embodiments, filtration of the cannabinoid concrete solution is through a filter of about 75 microns or finer. In certain embodiments, filtration of the cannabinoid concrete solution is through a filter of about 15 microns or larger. In certain embodiments, the filtration is through a filter of between about 15 microns and about 75 microns. In certain embodiments, the impurities are separated by precipitation. In certain embodiments, the precipitation is performed at about −100° C., −75° C., −50° C., −25° C., −10° C., 0° C., 5° C., 10° C., 15° C., 20° C., 25° C., or any range in between. In certain embodiments, the precipitation is performed at less than about −100° C., −75° C., −50° C., −25° C., −10° C., 0° C., 5° C., 10° C., 15° C., 20° C., or 25° C. In certain embodiments, the precipitation is performed at not lower than about −75° C., −50° C., −25° C., −10° C., 0° C., 5° C., 10° C., 15° C., 20° C., or 25° C.

### Method of Delivering a Cannabinoid

Certain aspects are drawn to methods of delivering a cannabinoid to a subject. In certain embodiments a method comprises placing an edible product comprising a cannabinoid-bonded polymer composition described anywhere herein into the mouth of the subject. In certain embodiments, once placed into the mouth, whether chewed, sucked, or swallowed, etc., a cannabinoid in the cannabinoid-bonded polymer composition is absorbed sublingually, mucosally, or sublingually and mucosally. In certain embodiments, the edible product is a chewing or bubble gum. In certain embodiments, the method produces psychoactive symptoms associated with cannabinoids in the subject.

The following examples of specific aspects are offered for illustrative purposes only, and are not intended to limit the scope of the present disclosure in any way.

### EXAMPLES

### Exemplary Methods of Preparing a Cannabinoid-Bonded Polymer Composition

### Example 1

1. Place a cannabinoid extraction in an alcohol or nontoxic hydrocarbon solvent (useful solvents include anhydrous ethanol, methyl ether, or butane) to dissolve the cannabinoid extraction at a temperature of from about 13° C. to about 82° C., depending on the solvent, the method of cannabinoid extraction used, and the concentration of the specific cannabinoids in the cannabinoid concentrate.

2. Cool the solvent solution (cannabinoid solvent absolute) to a temperature of from about 10° C. to about −125° C.

3. Separate the lipids, waxes, polymers, and plant materials (e.g., methyl cellulose) from the cannabinoid solvent absolute.

Methods of separation can include: (i) pouring a solution through a filter (micron rating dependent on solvent and temperature) or (ii) cool the solution and let precipitate to settle to the bottom and carefully decant the cannabinoid solvent absolute from the impurities.

4. Remove the solvent from the cannabinoid solvent absolute to produce a cannabinoid absolute. The solvent can be removed by heating, for example, heating the solvent to between 30° C. and 90° C., dependent on the method of cannabinoid extraction used to produce the cannabinoid concentrate and the concentration of specific cannabinoids in the cannabinoid solvent absolute.

5. Strip terpenes and decarboxylate cannabinoids by heating the cannabinoid absolute. Heat the cannabinoid absolute at a temperature of from about 65° C. to about 125° C., depending on the terpene spectrum and concentration of terpenes in the cannabinoid absolute. Heat for a time of from about 10 minutes to about 128 hours, depending on the temperature and the concentration of terpenes and terpene spectrum in the absolute.

6. Create a polymer-engrossing solution by dissolving a pharmaceutical-grade shellac into a solvent.

7. Dissolve the terpene stripped and decarboxylated cannabinoid absolute into the polymer-engrossing solvent. The temperature of the solvent is from a temperature of about 30° C. to about 110° C., depending on the solvent used to create the polymer-engrossing solvent.

The resultant solution is a cannabinoid-bonded polymer composition which can be stored, e.g., in a sealed, non-reactive container to prevent evaporation.

### Example 2

1. Place butane hash oil (BHO) into 350 mg of anhydrous ethanol at 24.5° C. for 45 minutes until completely dissolved.

2. Cool the resulting solution to 5° C. for 160 minutes.

3. Pour the solution through a 22 micron filter to remove waxes, lipids, and polymers.

4. Place the resultant solution (a dewaxed, winterized cannabinoid ethanol solution) in a vacuum oven at 22.15° C. and −22.916 Hg for 6.5 hours. This process removes 97.4% of the ethanol from the solution.

5. Place the remaining solution in an oven and heat to 136.6-145.2° C. for 1.25 hours. This process removes the remaining ethanol.

6. Decrease the temperature to 127.8° C. for 2.2 hours. This process strips the terpenes, terpenoids and other volatile compounds from the solution. This process also decarboxylates the remaining cannabinoids.

7. Remove the solution (“stripped and decarboxylated cannabinoid absolute”) from the oven and allow the solution to cool to 45.1° C.

8. While the solution is cooling, dissolve 2,500 mg of pharmaceutical-grade shellac into 250 ml of anhydrous ethanol at 89.9° C. for 21 minutes.

9. Once it has cooled, dissolve the stripped and decarboxylated cannabinoid absolute into the shellac and ethanol solution at 99.7° C. for 45 minutes.

10. Pour the resultant solution into a glass vessel, seal the vessel, and allow the vessel to cool to 29° C.

Once cooled, the product is a non-aromatic stable food additive with psychoactive cannabinoids that is ready for use.

Optionally, add 3 ml of terpenes (such as collected via distillation in a separate process) to 250 ml of the product. This encourages faster sublingual and mucosal absorption without compromising the integrity of the cannabinoids in the product.

### Example 3

Xylitol and Maltitol can be used to control the recrystallization of ethanol sugars that contribute to the stability and hardness of the final product (e.g., gum coating).

Ingredients: Vox (stripped and decarboxylated cannabinoid absolute), Titanium Dioxide, Maltitol, Xylitol, Shellac, 311 (maltodextrin, cellulose gum, and corn starch), Quick Crunch (maltodextrin, cellulose gum, and corn starch), Gum Base, Gum Arabic (gum acacia), Spearmint Oil, Cyclodextrin, Anhydrous Ethanol, Food Coloring (i.e., Blue and Yellow).

Combo 1=Vox+Titanium Dioxide+Xylitol+Shellac

Combo 2=Vox+Titanium Dioxide+Maltitol+Shellac

Combo 3=Vox+Titanium Dioxide+Maltitol+Xylitol+Shellac

Combo 4=Vox+Titanium Dioxide+Xylitol+Maltitol

Combo 5=Vox+Titanium Dioxide+Shellac

Combo 6=Vox+Xylitol+Shellac

Combo 7=Vox+Maltitol+Shellac

Combo 8=Vox+Xylitol+Maltitol+Shellac

Combo 9=Vox+Xylitol+Titanium Dioxide+Shellac

Combo 10=Vox+Maltitol+Titanium Dioxide+Shellac

Combo 11=Vox+Maltitol+Xylitol+Titanium Dioxide+Shellac

Combo 12=Vox+Maltitol+Shellac

Combo 13=Vox+Xylitol+Shellac

Combo 14=Vox+Xylitol+Maltitol+Shellac

What is claimed is:

1. A method of producing a cannabinoid-bonded polymer composition, the method comprising:
a) removing at least about 90% of the solvent from, stripping at least of portion of the terpenes from, and decarboxylating at least a portion of the cannabinoids contained in a cannabinoid solvent absolute obtained from a dewaxed cannabinoid concentrate concrete, to produce a stripped and decarboxylated cannabinoid absolute; and
b) dissolving the stripped and decarboxylated cannabinoid absolute in a polymer-engrossing solution comprising a food grade polymer and a solvent;
thereby producing a cannabinoid-bonded polymer composition.

2. The method of producing a cannabinoid-bonded polymer composition of claim 1, wherein the cannabinoid solvent absolute obtained from a dewaxed cannabinoid concentrate concrete is obtained by:
i) dissolving a cannabinoid concentrate concrete in a solvent at a temperature of between about 13° C. to about 82° C. to produce a cannabinoid concrete solution;
ii) cooling the cannabinoid concrete solution to a temperature of between about 10° C. and about −125° C.; and
iii) separating at least a portion of the impurities from the cooled cannabinoid concrete solution to produce a cannabinoid solvent absolute.

3. The method of producing a cannabinoid-bonded polymer composition of claim 1 or 2, wherein the solvent of the cannabinoid solvent absolute has a boiling point of from about 20° C. to about 100° C.

4. The method of producing a cannabinoid-bonded polymer composition of any one of claims 1 to 3, wherein the solvent of the cannabinoid solvent absolute is an alcohol or nontoxic hydrocarbon solvent.

5. The method of producing a cannabinoid-bonded polymer composition of any one of claims 1 to 3, wherein the solvent of the cannabinoid solvent absolute is selected from the group consisting of methyl ether, butane, hexane, propane, ethanol, and carbon dioxide.

6. The method of producing a cannabinoid-bonded polymer composition of claim 5, wherein the solvent of the cannabinoid solvent absolute is selected from the group consisting of anhydrous ethanol, methyl ether, and butane.

7. The method of producing a cannabinoid-bonded polymer composition of any one of claims 2 to 6, wherein the impurities are separated by filtration.

8. The method of producing a cannabinoid-bonded polymer composition of claim 7, wherein the filtration is through a filter of about 75 microns or finer.

9. The method of producing a cannabinoid-bonded polymer composition of claim 7, wherein the filtration is through a filter of between about 15 microns and about 75 microns.

10. The method of producing a cannabinoid-bonded polymer composition of any one of claims 2 to 6, wherein the impurities are separated by precipitation.

11. The method of producing a cannabinoid-bonded polymer composition of claim 10, wherein the precipitation is performed at temperature of between about −100° C. and about 15° C.

12. The method of producing a cannabinoid-bonded polymer composition of any one of claims 1 to 11, wherein step a) comprises the separate steps of:
a)(i) first removing the solvent from the cannabinoid solvent absolute to form a cannabinoid absolute; and
a)(ii) then stripping the terpenes from the cannabinoid absolute and decarboxylating the cannabinoids in the cannabinoid absolute to produce a stripped and decarboxylated cannabinoid ab solute.

13. The method of producing a cannabinoid-bonded polymer composition of any one of claims 1 to 12, wherein removing the solvent comprises heating the cannabinoid solvent absolute to a temperature of at least about 30° C. to about the boiling point of the cannabinoid in the cannabinoid solvent absolute with the lowest boiling point.

14. The method of producing a cannabinoid-bonded polymer composition of any one of claims 1 to 12, wherein removing the solvent comprises heating the cannabinoid solvent absolute to a temperature of between about 30° C. to about 90° C.

15. The method of producing a cannabinoid-bonded polymer composition of any one of claims 1 to 14, wherein stripping the terpenes and decarboxylating the cannabinoids is performed at a temperature of between about 65° C. and about the boiling point of the cannabinoid in the cannabinoid solvent absolute with the lowest boiling point.

16. The method of producing a cannabinoid-bonded polymer composition of any one of claims 1 to 14, wherein stripping the terpenes and decarboxylating the cannabinoids is performed at a temperature of between about 65° C. and about 125° C.

17. The method of producing a cannabinoid-bonded polymer composition of any one of claims 1 to 16, wherein stripping the terpenes and decarboxylating the cannabinoids is done for a duration of time of between about 10 minutes to about 128 hours or of between about 7 hours to about 24 hours.

18. The method of producing a cannabinoid-bonded polymer composition of any one of claims 1 to 17, wherein at least about 50% of the terpenes are stripped from those present in the cannabinoid solvent absolute.

19. The method of producing a cannabinoid-bonded polymer composition of any one of claims 1 to 18, wherein at least about 80% of the cannabinoids present in the stripped and decarboxylated cannabinoid absolute are decarboxylated.

20. The method of producing a cannabinoid-bonded polymer composition of any one of claims 1 to 19, wherein the stripped and decarboxylated cannabinoid absolute is dissolved in the polymer engrossing solution at a temperature of between about 30° C. and 110° C.

21. The method of producing a cannabinoid-bonded polymer composition of any one of claims 1 to 20, further comprising the step of adding one or more terpenes to the cannabinoid-bonded polymer composition.

22. The method of producing a cannabinoid-bonded polymer composition of claim 21, wherein the one or more terpenes is selected from the group consisting of menthol, menthone, menthoxypropanediol, menthylacetate, myrcenol, citronellal, senchone, and thioterpineol

23. The method of producing a cannabinoid-bonded polymer composition of any one of claims 1 to 22, wherein the cannabinoid-bonded polymer composition exhibits psychoactive cannabinoid activity.

24. The method of producing a cannabinoid-bonded polymer composition of any one claims 1 to 23, wherein the cannabis odor of the cannabinoid-bonded polymer composition is less than that of the starting cannabinoid concentrate concrete containing the same concentration of cannabinoids, as determined by a blind odor test group containing a statistically significant number of subjects.

25. The method of producing a cannabinoid-bonded polymer composition of any one of claims 1 to 24, wherein the cannabis odor of the cannabinoid-bonded polymer composition, containing a cannabinoid concentration of at least about 1% by weight, is imperceptible on average to subjects in an odor test group, containing a statistically significant number of subjects, who are unaware that the cannabinoid-bonded polymer composition contains cannabinoids.

26. The method of producing a cannabinoid-bonded polymer composition of any one of claims 1 to 24, wherein the cannabis odor of the cannabinoid-bonded polymer composition, containing a cannabinoid concentration of at least about 1% by weight, is imperceptible to at least about 60% of subjects in an odor test group, containing a statistically significant number of subjects, who are unaware that the cannabinoid-bonded polymer composition contains cannabinoids.

27. The method of producing a cannabinoid-bonded polymer composition of any one claims 1 to 26, wherein the cannabis taste of the cannabinoid-bonded polymer composition is less than in the starting cannabinoid concentrate concrete containing the same concentration of cannabinoids, as determined by a blind taste test group containing a statistically significant number of subj ects.

28. The method of producing a cannabinoid-bonded polymer composition of any one of claims 1 to 27, wherein the cannabis taste of the cannabinoid-bonded polymer composition, containing a cannabinoid concentration of at least about 1% by weight, is imperceptible on average to subjects in a taste test group, containing a statistically significant number of subjects, who are unaware whether the cannabinoid-bonded polymer composition contains cannabinoids.

29. The method of producing a cannabinoid-bonded polymer composition of any one of claims 1 to 27, wherein the cannabis taste of the cannabinoid-bonded polymer composition, containing a cannabinoid concentration of at least about 1% by weight, is imperceptible to at least about 60% of subjects in a taste test group, containing a statistically significant number of subjects, who are unaware whether the cannabinoid-bonded polymer composition contains cannabinoids.

30. The method of producing a cannabinoid-bonded polymer composition of any one claims 1 to 29, wherein the cannabis odor of an edible product that exhibits psychoactive cannabinoid activity, comprising the cannabinoid-bonded polymer composition, is imperceptible on average or imperceptible to at least about 60% to subjects in an odor test group, containing a statistically significant number of subjects, who are unaware whether the edible product contains cannabinoids.

31. The method of producing a cannabinoid-bonded polymer composition of any one claims 1 to 30, wherein the cannabis taste of an edible product that exhibits psychoactive cannabinoid activity, comprising the cannabinoid-bonded polymer composition, is imperceptible on average or imperceptible to at least about 60% of subjects in a taste test group, containing a statistically significant number of subjects, who are unaware whether the edible product contains cannabinoids.

32. The method of producing a cannabinoid-bonded polymer composition of any one of claims 1 to 31, wherein the solvent of the polymer engrossing solution comprises ethanol.

33. The method of producing a cannabinoid-bonded polymer composition of any one of claims 1 to 32, wherein the food grade polymer of the polymer engrossing solution comprises shellac.

34. The method of producing a cannabinoid-bonded polymer composition of any one of claims 1 to 33, wherein the cannabinoid-bonded polymer composition comprises pharmaceutical lactose and/or xylitol.

35. The method of producing a cannabinoid-bonded polymer composition of any one of claims 1 to 34, wherein the cannabinoid-bonded polymer composition comprises from about 0.1% to about 22% by weight of cannabinoids.

36. The method of producing a cannabinoid-bonded polymer composition of claim 35, wherein the cannabinoid-bonded polymer composition comprises from about 2% to about 18% by weight of cannabinoids.

37. A cannabinoid-bonded polymer composition made by the method of any one of claims 1 to 36.

38. The cannabinoid-bonded polymer composition of claim 37 comprising about 0.1% to about 22% by weight of cannabinoids.

39. The cannabinoid-bonded polymer composition of claim 38 comprising about 2% to about 18% by weight of cannabinoid.

40. The cannabinoid-bonded polymer composition of any one of claims 37 to 39 comprising ethanol.

41. The cannabinoid-bonded polymer composition of any one of claims 37 to 40 comprising shellac.

42. The cannabinoid-bonded polymer composition of any one of claims 37 to 41 comprising pharmaceutical lactose and/or xylitol.

43. A cannabinoid-bonded polymer composition comprising about 0.1% to about 22% by weight of a cannabinoid, a food grade polymer, and a solvent.

44. The cannabinoid-bonded polymer composition of claim 43 comprising about 2% to about 18% by weight of a cannabinoid.

45. The cannabinoid-bonded polymer composition of claim 43 or 44, wherein the solvent is ethanol.

46. The cannabinoid-bonded polymer composition of any one of claims 43 to 45, wherein the food grade polymer is shellac.

47. The cannabinoid-bonded polymer composition of any one of claims 43 to 46, wherein the cannabinoid-bonded polymer composition comprises pharmaceutical lactose and/or xylitol.

48. The cannabinoid-bonded polymer composition of any one of claims 43 to 47, wherein the composition contains a cannabinoid concentration of at least about 1% by weight and the cannabis odor of the composition is imperceptible on average to or is imperceptible to at least about 60% of subjects in an odor test group, containing a statistically significant number of subjects, who are unaware that the cannabinoid-bonded polymer composition contains cannabinoids.

49. The cannabinoid-bonded polymer composition of any one of claims 43 to 48, wherein the composition contains a cannabinoid concentration of at least about 1% by weight and the cannabis taste of the composition is imperceptible on average to or is imperceptible to at least about 60% of subjects in a taste test group, containing a statistically significant number of subjects, who are unaware whether the cannabinoid-bonded polymer composition contains cannabinoids.

50. The cannabinoid-bonded polymer composition of any one of claims 43 to 49, wherein the cannabis odor of an edible product that exhibits psychoactive cannabinoid activity, comprising the cannabinoid-bonded polymer composition, is imperceptible on average to or is imperceptible to at least about 60% of subjects in an odor test group, containing a statistically significant number of subjects, who are unaware that the edible product contains cannabinoids.

51. The cannabinoid-bonded polymer composition of any one of claims 43 to 50, wherein the cannabis taste of an edible product that exhibits psychoactive cannabinoid activity, comprising the cannabinoid-bonded polymer composition, is imperceptible on average to or is imperceptible to at least about 60% of subjects in a taste test group, containing a statistically significant number of subjects, who are unaware whether the edible product contains cannabinoids.

52. A method of delivering a cannabinoid to a subject, the method comprising placing an edible product that comprises a cannabinoid-bonded polymer composition of any one of claims 37 to 51 into the mouth of the subject.

53. The method of delivering a cannabinoid to a subject of claim 52, wherein a cannabinoid in the cannabinoid-bonded polymer composition is absorbed sublingually, mucosally, or sublingually and mucosally.

54. The method of delivering a cannabinoid to a subject of claim 52 or 53, wherein the edible product is a chewing gum.

55. The method of delivering a cannabinoid to a subject of any one of claim 52 or 54, wherein the method produces psychoactive symptoms associated with cannabinoids in the subj ect.

56. An edible product comprising the cannabinoid-bonded polymer composition of any one of claims 37 to 51.

57. The edible product of claim 56, wherein product has psychoactive activity associated with cannabinoids.

58. The edible product of claim 56 or 57, wherein the cannabis odor from the cannabinoid-bonded polymer composition in the edible product is imperceptible on average to or imperceptible to at least about 60% of subjects in an odor test group, containing a statistically significant number of subjects, who are unaware whether the edible product contains cannabinoids.

59. The edible product of claim 56 or 58, wherein the cannabis taste from the cannabinoid-bonded polymer composition in the edible product is imperceptible on average to or imperceptible to at least about 60% of subjects in a taste test group, containing a statistically significant number of subjects, who are unaware whether the edible composition contains cannabinoids.

60. The edible product of claim of any one of claims 56 to 59, wherein the product is a chewing gum.
